(19) United States
(12) Patent Application Publication
(10) Pub. No.: US 2025/0388766 A1
(43) Pub. Date: Dec. 25, 2025

(54) METAL OXIDE DISPERSON AND METHOD FOR MANUFACTURING METAL OXIDE FILM USING SAME

(71) Applicant: TOKYO OHKA KOGYO CO., LTD., Kanagawa (JP)

(72) Inventors: Kenichi YAMAUCHI, Kanagawa (JP); Kunihiro NODA, Kanagawa (JP); Michihito TAKAHASHI, Kanagawa (JP); Saeko HARAGUCHI, Kanagawa (JP); Atsushi YAMANOUCHI, Kanagawa (JP); Dai SHIOTA, Kanagawa (JP)

(21) Appl. No.: 18/877,518

(22) PCT Filed: Jun. 20, 2023
(86) PCT No.: PCT/JP2023/022680

§ 371 Date: Dec. 20, 2024

(30) Foreign Application Priority Data
Jun. 23, 2022 (JP) 2022-101070

(51) Int. Cl. C09D 5/34; C09D 1/00; C09D 7/63

(52) U.S. Cl. CPC C09D 5/34; C09D 1/00; C09D 7/63

(57) ABSTRACT

Provided is a metal oxide dispersion having excellent gap-fill performance, and a method of manufacturing a metal oxide film using the same. A metal oxide dispersion according to a first aspect of the present invention contains carboxylic acid having 9 or more carbon atoms, metal oxide nanoparticles surface-treated with a capping agent, and a solvent. A metal oxide dispersion according to a second aspect of the present invention contains carboxylic acid having a boiling point of 250° C. or higher at an atmospheric pressure, metal oxide nanoparticles surface-treated with a capping agent, and a solvent. A method of manufacturing a metal oxide film according to a third aspect of the present invention comprises: a step for forming a coating film formed of the metal oxide dispersion described above; and a step for heating the coating film at a temperature of 165° C. or higher.

### TECHNICAL FIELD

The present invention relates to a metal oxide dispersion and a method for manufacturing a metal oxide film using the same.

### BACKGROUND ART

Metal oxide films used for a hard mask or the like can be formed by forming a film of a metal oxide dispersion by a liquid phase coating method such as a spin coating method or an inkjet method. As the metal oxide dispersion, for example, a coating composition is known, which includes an organic solvent, metal oxide nanoparticles dispersed in the organic solvent, and a high carbon polymer having a specific structure dissolved in the solvent (see Patent Document 1).

### CITATION LIST

### Patent Document

Patent Document 1: Japanese Unexamined Patent Application (Translation of PCT Application), Publication No. 2020-503409

### DISCLOSURE OF THE INVENTION

### Problems to be Solved by the Invention

When forming a metal oxide film on a substrate having a step, such as a substrate having a hole, a trench, or the like, or a substrate provided with another member, the metal oxide film is required to have excellent gap-fill characteristics for filling irregularities on the substrate.

The present invention has been made in view of such conventional circumstances, and an object of the present invention is to provide a metal oxide dispersion having excellent gap-fill characteristics and a method for manufacturing a metal oxide film using the same.

### Means for Solving the Problems

The present inventors have conducted intensive studies to solve the above problems. As a result, the present inventors have found that the above problems can be solved by a metal oxide dispersion containing a carboxylic acid having 9 or more carbon atoms or a carboxylic acid having a boiling point of 250° C. or more at atmospheric pressure, metal oxide nanoparticles surface-treated with a capping agent, and a solvent, thereby arriving at completion of the present invention. Specifically, the present invention provides the following.

A first aspect of the present invention relates to a metal oxide dispersion including:
a carboxylic acid having 9 or more carbon atoms;
metal oxide nanoparticles surface-treated with a capping agent, and a solvent.

A second aspect of the present invention relates to a metal oxide dispersion including:
a carboxylic acid having a boiling point of 250° C. or higher at atmospheric pressure;
metal oxide nanoparticles surface-treated with a capping agent; and
a solvent.

A third aspect of the present invention relates to a method for manufacturing a metal oxide film including: a coating film forming step of forming a coating film including the metal oxide dispersion; and a heating step of heating the coating film at a temperature of 165° C. or higher.

### Effects of the Invention

According to the present invention, it is possible to provide a metal oxide dispersion having excellent gap-fill characteristics and a method for manufacturing a metal oxide film using the same.

### Preferred Mode for Carrying Out the Invention

### <Metal Oxide Dispersion>

The metal oxide dispersion as described in the first aspect of the present invention contains a carboxylic acid having 9 or more carbon atoms, metal oxide nanoparticles surface-treated with a capping agent, and a solvent. The metal oxide dispersion as described in the second aspect of the present invention contains a carboxylic acid having a boiling point of 250° C. or higher at atmospheric pressure, metal oxide nanoparticles surface-treated with a capping agent, and a solvent. The metal oxide dispersion according to the present invention has excellent gap-fill characteristics.

In a solid content of the metal oxide dispersion, a ratio of an inorganic component mass to a total of the inorganic component mass and an organic component mass is 25 mass % or more, preferably 30 mass % or more, and more preferably 40 mass % or more. When the ratio is within the above range, the ratio of the inorganic component mass can be set high, and as a result, the ratio of the inorganic component mass can be easily improved in the metal oxide dispersion. The upper limit of the ratio is not particularly limited, and may be 90% by mass, 80% by mass, or 75% by mass. [Carboxylic Acids]

The metal oxide dispersion as described in the first aspect of the present invention includes a carboxylic acid having 9 or more carbon atoms. When the metal oxide dispersion contains a carboxylic acid having 9 or more carbon atoms or a carboxylic acid having a boiling point of 250° C. or more at atmospheric pressure, the metal oxide dispersion tends to have improved gap-fill characteristics. The carboxylic acids having 9 or more carbon atoms may be used alone or in combination of two or more types thereof.

In the first aspect of the present invention, the number of carbon atoms of the carboxylic acid is preferably 9 to 35, more preferably 10 to 24, and still more preferably 12 to 18. When the number of carbon atoms of a carboxylic acid is within the above range, the gap-fill characteristics tend to be improved.

The metal oxide dispersion as described in the second aspect of the present invention contains a carboxylic acid having a boiling point of 250° C. or higher at atmospheric pressure. When the metal oxide dispersion contains a carboxylic acid having a boiling point of 250° C. or higher at atmospheric pressure, the metal oxide dispersion tends to have improved gap-fill characteristics. The carboxylic acids having a boiling point of 250° C. or higher at atmospheric pressure may be used alone or in combination of two or more types thereof.

In the second aspect of the present invention, the boiling point of the carboxylic acid at atmospheric pressure is preferably 260 to 500° C., more preferably 275 to 470° C., and still more preferably 290 to 440° C. When the boiling point of the carboxylic acid at atmospheric pressure is within the above range, the gap-fill characteristics tend to be improved.

In a case in which the capping agent to be described below contains a carboxylic acid, in the metal oxide dispersion according to the present invention, the carboxylic acid is present in the capping agent as well as being present separately from the metal oxide nanoparticles. The carboxylic acid having 9 or more carbon atoms and the carboxylic acid having a boiling point of 250° C. or more at atmospheric pressure refer to a carboxylic acid present in the metal oxide dispersion independently of the metal oxide nanoparticles. That is, the carboxylic acid having 9 or more carbon atoms and the carboxylic acid having a boiling point of 250° C. or more at atmospheric pressure refer to a carboxylic acid present in the metal oxide dispersion in a free state.

Specific examples of the carboxylic acid having 9 or more carbon atoms or the carboxylic acid having a boiling point of 250° C. or more at atmospheric pressure include linear saturated fatty acids having 9 or more, preferably 9 to 35, more preferably 10 to 24, still more preferably 12 to 18 carbon atoms, such as pelargonic acid, capric acid, undecylic acid, lauric acid, tridecylic acid, myristic acid, pentadecylic acid, palmitic acid, malgaric acid, stearic acid, nonadecylic acid, arachidic acid, heneicosylic acid, behenic acid, tricosylic acid, lignoceric acid, etc.; linear unsaturated fatty acids having 9 or more, preferably 9 to 35, more preferably 10 to 24, and even more preferably 12 to 18 carbon atoms, such as α-linolenic acid (ALA), stearidonic acid (STD), eicosatetraenoic acid (ETA), eicosapentaenoic acid (EPA), docosapentaenoic acid (DPA) (curpanodonic acid), docosahexaenoic acid (DHA), linoleic acid, γ-linolenic acid (GLA), dihomo-γ-linolenic acid (DGLA), arachidonic acid (ARA), docosatetraenoic acid, docosapentaenoic acid (osbond acid), palmitoleic acid, vaccenic acid, pauric acid, oleic acid, elaidic acid, erucic acid, nervonic acid, sapienic acid, etc.; branched fatty acids having 9 or more, preferably 9 to 35, more preferably 10 to 24, and even more preferably 12 to 18 carbon atoms, such as isopalmitic acid, isostearic acid, 4-methyl-n-octanoic acid, 4-methyl-n-nonanoic acid, 2-hexadecyloctadecanoic acid, etc.; aromatic carboxylic acids having 9 or more, preferably 9 to 35, more preferably 10 to 24, and even more preferably 12 to 18 carbon atoms, such as alkylbenzoic acid having 9 or more carbon atoms, alkyloxybenzoic acid having 9 or more carbon atoms, alkylphthalic acid, alkyloxyphthalic acid, monoalkyl isophthalate, monoalkyl terephthalate, alkoxycinnamic acid, dialkoxyether cinnamic acid, 4-(4-propylphenyl)benzoic acid, 4-(4-heptylphenyl)benzoic acid, 4′-decyloxybiphenyl-4-carboxylic acid, other 4′-alkyloxybiphenyl-4-carboxylic acids, etc.; alkyl polycarboxylic acids having 9 or more, preferably 9 to 35, more preferably 10 to 24, and even more preferably 12 to 18 carbon atoms, such as n-octylsuccinic acid, spiculisporic acid, etc.; alkylsarcosines having 9 or more, preferably 9 to 35, more preferably 10 to 24, and even more preferably 12 to 18 carbon atoms, such as laurylsarcosine, etc.; acylsarcosines having 9 or more, preferably 9 to 35, more preferably 10 to 24, and even more preferably 12 to 18 carbon atoms, such as oleoylsarcosine, etc.; hydroxy acids having 9 or more, preferably 9 to 35, more preferably 10 to 24, and even more preferably 12 to 18 carbon atoms, such as 12-hydroxystearic acid, aleuritic acid, 2-hydroxypalmitic acid, etc.; and carboxylic acids containing ethereal oxygen or thioethereal sulfur in the carbon chain and having 9 or more, preferably 9 to 35, more preferably 10 to 24, and even more preferably 12 to 18 carbon atoms, such as 3-(dodecylthio) propionic acid, etc. From the viewpoint of improving gap-fill characteristics or the like, lauric acid, stearic acid, n-octylsuccinic acid, 4-(hexyloxy)benzoic acid, and 12-hydroxystearic acid are preferable.

An amount of the carboxylic acid which has 9 or more carbon atoms to be used, or the carboxylic acid having a boiling point of 250° C. or more at atmospheric pressure is not particularly limited, and is preferably 1 to 20% by mass, more preferably 3 to 15% by mass, and still more preferably 5 to 12% by mass with respect to a total of components other than the solvent in the metal oxide dispersion. When the amount of the carboxylic acid to be used is within the above range, the gap-fill characteristics of the metal oxide dispersion tend to be improved.

[Metal Oxide Nanoparticles Surface-Treated with Capping Agent]

The metal oxide dispersion includes metal oxide nanoparticles surface-treated with a capping agent. In the present specification, the metal oxide nanoparticles consist of a metal oxide and do not contain a capping agent. The metal oxide nanoparticles surface-treated with the capping agent may be used alone or in combination of two or more types thereof. When the metal oxide dispersion contains metal oxide nanoparticles surface-treated with a capping agent, the metal oxide dispersion tends to have improved gap-fill characteristics.

An average particle diameter of metal oxide nanoparticles is preferably 5 nm or less, more preferably 4 nm or less, and still more preferably 3 nm or less. The lower limit of the average particle diameter of the metal oxide nanoparticles is not particularly limited, and may be, for example, 0.5 nm or more, 1 nm or more, or 2 nm or more. When the average particle diameter of the metal oxide nanoparticles is within the above range, the gap-fill characteristics of the metal oxide dispersion tend to be improved. In the present specification, the average particle diameter of the metal oxide nanoparticles refers to a value obtained by performing XRD measurement with an X-ray diffractometer (SmartLab, manufactured by Rigaku), analyzing the obtained result with PDXL as an attached software, and then performing the Halder-Wagner method.

The average particle diameter of the metal oxide nanoparticles surface-treated with the capping agent is preferably 10 nm or less, more preferably 8 nm or less, and still more preferably 6 nm or less. The lower limit is not particularly limited, and may be, for example, 0.5 nm or more, 1 nm or more, or 2 nm or more. In the present specification, the average particle diameter of the metal oxide nanoparticles surface-treated with the capping agent refers to a value measured using a dynamic light scattering (DLS) apparatus such as Malvern Zetasizer Nano S.

The metal contained in the metal oxide nanoparticles is not particularly limited, and examples thereof include zinc, yttrium, hafnium, zirconium, lanthanum, cerium, neodymium, gadolinium, holmium, lutetium, tantalum, titanium, silicon, aluminum, antimony, tin, indium, tungsten, copper, vanadium, chromium, niobium, molybdenum, ruthenium, rhodium, rhenium, iridium, germanium, gallium, thallium, and magnesium. From the viewpoint of the film-forming property, stability and the like, hafnium, zirconium, titanium, and tin are preferable, and zirconium is more preferable. The above metals may be used alone or in combination of two or more types thereof.

A metal oxide nanoparticle may be composed of a metal atom and an oxygen atom, or may be composed of a metal atom, an oxygen atom, and an atom other than the metal atom and the oxygen atom. Examples of the atom other than the metal atom and the oxygen atom include a nitrogen atom. Therefore, a metal oxide nanoparticle may consist of a metal oxide or may consist of a metal oxynitride or the like.

In the metal oxide dispersion according to the present invention, it is presumed that a part or all of the surface of a metal oxide nanoparticle is covered with a capping agent. The capping agent includes at least one selected from the group consisting of alkoxysilanes, phenols, alcohols, carboxylic acids, and carboxylic acid halides. In the metal oxide dispersion according to the present invention, when the metal oxide nanoparticles are surface-treated with a capping agent, dispersibility of the metal oxide nanoparticles in a solvent is easily stabilized, and gap-fill characteristics of the metal oxide dispersion tend to be improved.

Specific examples of the capping agent include alkoxysilanes such as n-propyltrimethoxysilane, n-propyltriethoxysilane, n-octyltrimethoxysilane, n-octyltriethoxysilane, n-dodecyltrimethoxysilane, n-dodecyltriethoxysilane, n-hexadecyltrimethoxysilane, n-hexadecyltriethoxysilane, n-octadecyltrimethoxysilane, n-octadecyltriethoxysilane, phenyltrimethoxysilane, phenyltriethoxysilane, phenethylphenyltrimethoxysilane, phenethylethyltriethoxysilane, 3-{2-methoxy [poly(ethyleneoxy)]}propyltrimethoxysilane, 3-{2-methoxy [poly(ethyleneoxy)]}propyltriethoxysilane, 3-{2-methoxy [tri (ethyleneoxy)]}propyltrimethoxysilane, 3-{2-methoxy [tri (ethyleneoxy)]}propyltriethoxysilane, vinyltrimethoxysilan, vinyltriethoxysilane, allyltrimethoxysilane, allyltriethoxysilane, 1-hexenyltrimethoxysilane, 1-hexenyltriethoxysilane, 1-octenyltrimethoxysilane, 1-octenyltriethoxysilane, 3-aminopropyltrimethoxysilane, 3-aminopropyltriethoxysilane, 3-mercaptopropyltrimethoxysilane, 3-mercaptopropyltriethoxysilane, 3-acryloyloxypropyltrimethoxysilane, 3-acryloylpropyltriethoxysilane, 3-methacryloyloxypropyltrimethoxysilane, 3-methacryloyloxypropyltriethoxysilane, 3-isocyanatopropyltrimethoxysilane, 3-isocyanatopropyltriethoxysilane, 3-glycidoxypropyltrimethoxysilane, 3-glycidoxypropyltriethoxysilane, etc.; phenols such as phenol, etc.; unsaturated group-free alcohols such as ethanol, n-propanol, isopropanol, n-butanol, n-heptanol, n-hexanol, n-octanol, n-dodecyl alcohol, n-octadecanol, benzyl alcohol, triethylene glycol monomethyl ether, etc.; unsaturated group-containing alcohols such as 2-hydroxyethyl (meth)acrylate, 3-hydroxypropyl(meth) acrylate, allyl alcohol, oleyl alcohol, ethylene glycol monoallyl ether, propylene glycol monoallyl ether, 3-allyloxypropanol, etc.; acids such as octanoic acid, acetic acid, propionic acid, 2-[2-(methoxyethoxy) ethoxy]acetic acid, oleic acid, lauric acid, benzoic acid, 2-acryloyloxyethyl succinic acid, 2-acryloyloxyethyl phthalic acid, etc.; and acid halides of these acids such as acid chlorides of these acids. Preferable examples thereof include alkoxysilanes, unsaturated group-containing alcohols, or compounds exemplified as acids.

An amount of the capping agent used in the surface treatment of the metal oxide nanoparticles with the capping agent is not particularly limited. Preferably, an amount of capping agent sufficient to react with substantially all of the hydroxy groups on the surface of the metal oxide nanoparticles is used.

A content of the metal oxide nanoparticles in the metal oxide dispersion is not particularly limited as long as the object of the present invention is not impaired, and is preferably 5 mass % or more and 99 mass % or less, more preferably 30 mass % or more and 98 mass % or less, and still more preferably 60 mass % or more and 97 mass % or less, with respect to a total of components other than the solvent in the metal oxide dispersion. When the content is within the above range, the gap-fill characteristics of the metal oxide dispersion tend to be improved. The content of metal oxide nanoparticles includes the content of the capping agent present on the surface of the metal oxide nanoparticles.

A total content of the carboxylic acid and the metal oxide nanoparticles surface-treated with the capping agent in the metal oxide dispersion is not particularly limited as long as the object of the present invention is not impaired, and is preferably 90% by mass or more and 100% by mass or less, more preferably 95% by mass or more and 100% by mass or less, and may be 100% by mass with respect to the total of the components other than the solvent in the metal oxide dispersion.

### [Solvents]

The metal oxide dispersion according to the present invention contains a solvent for the purpose of adjusting coating properties, viscosity, and the like. As the solvent, an organic solvent is typically used. The type of organic solvent is not particularly limited as long as components contained in the metal oxide dispersion can be uniformly dissolved or dispersed.

Suitable examples of the organic solvent that can be used as the solvent include: (poly)alkylene glycol monoalkyl ethers such as ethylene glycol monomethyl ether, ethylene glycol monoethyl ether, ethylene glycol n-propyl ether, ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol monomethyl ether, propylene glycol monoethyl ether, propylene glycol mono-n-propyl ether, propylene glycol mono-n-butyl ether, dipropylene glycol monomethyl ether, dipropylene glycol monoethyl ether, dipropylene glycol mono-n-propyl ether, dipropylene glycol mono-n-butyl ether, tripropylene glycol monomethyl ether, tripropylene glycol monoethyl ether, etc.; (poly)alkylene glycol monoalkyl ether acetates such as ethylene glycol monomethyl ether acetate, ethylene glycol monoethyl ether acetate, diethylene glycol monomethyl ether acetate, diethylene glycol monoethyl ether acetate, propylene glycol monomethyl ether acetate, propylene glycol monoethyl ether acetate, etc.;
other ethers such as diethylene glycol dimethyl ether, diethylene glycol methyl ethyl ether, diethylene glycol diethyl ether, tetrahydrofuran, etc.; ketones such as methyl ethyl ketone, cyclohexanone, 2-heptanone, 3-heptanone, etc.; alkyl lactates such as methyl 2-ydroxypropionate, ethyl 2-hydroxypropionate, etc.; other esters such as ethyl 2-hydroxy-2-methylpropionate, methyl 3-methoxypropionate, ethyl 3-methoxypropionate, methyl 3-ethoxypropionate, ethyl 3-ethoxypropionate, ethyl ethoxyacetate, ethyl hydroxyacetate, methyl 2-hydroxy-3-methylbutanoate, 3-methyl-3-methoxybutyl acetate, 3-methyl-3-methoxybutyl propionate, ethyl acetate, n-propyl acetate, isopropyl acetate, n-butyl acetate, isobutyl acetate, n-pentyl formate, isopentyl acetate, n-butyl propionate, ethyl butyrate, n-propyl butyrate, isopropyl butyrate, n-butyl butyrate, methyl pyruvate, ethyl pyruvate, n-propyl pyruvate, methyl acetoacetate, ethyl acetoacetate, ethyl 2-oxobutanoate, etc.; aromatic hydrocarbons such as toluene, xylene, etc.; amides such as N-methylpyrrolidone, N, N-dimethylformamide, N, N-dimethylacetamide, etc. or the like. These organic solvents can be used alone or in combination of two or more types thereof.

An amount of the solvent to be used in the metal oxide dispersion according to the present invention is not particularly limited. From the viewpoint of coatability of the metal oxide dispersion, the amount of the solvent to be used is, for example, 30 to 99.9% by mass, more preferably 40 to 99.5% by mass, and still more preferably 50 to 99% by mass with respect to the entire metal oxide dispersion.

### [Surfactants]

The metal oxide dispersion according to the present invention may further contain a surfactant (surface modifier) in order to improve film-forming properties, coating properties, defoaming properties, leveling properties, and the like. The surfactants may be used alone or in combination of two or more types thereof. Examples of the surfactant include silicone surfactants, fluorine surfactants, and polymer wetting dispersants, and polymer wetting dispersants are particularly preferable from the viewpoint of improving film-forming properties.

Specific examples of the silicone surfactant include BYK-077, BYK-085, BYK-300, BYK-301, BYK-302, BYK-306, BYK-307, BYK-310, BYK-320, BYK-322, BYK-323, BYK-325, BYK-330, BYK-331, BYK-333, BYK-335, BYK-341, BYK-344, BYK-345, BYK-346, BYK-348, BYK-354, BYK-355, BYK-356, BYK-358, BYK-361, BYK-370, BYK-371, BYK-375, BYK-380, and BYK-390 (manufactured by BYK Chemie).

Specific examples of the fluorine-based surfactant include F-114, F-177, F-410, F-411, F-450, F-493, F-494, F-443, F-444, F-445, F-446, F-470, F-471, F-472SF, F-474, F-475, F-477, F-478, F-479, F-480SF, F-482, F-483, F-484, F-486, F-487, F-172D, MCF-350SF, TF-1025SF, TF-1117SF, TF-1026SF, TF-1128, TF-1127, TF-1129, TF-1126, TF-1130, TF-1116SF, TF-1131, TF-1132, TF-1027SF, TF-1441, and TF-1442 (manufactured by DIC Corporation); PF-636, PF-6320, PF-656, PF-6520 of polyfox series (manufactured by Omnova); or the like.

Specific examples of the polymer wetting dispersant include BYK-140, BYK-145, BYK-161, BYK-162, BYK-163, BYK-164, BYK-167, BYK-168, BYK-170, BYK-171, BYK-174, BYK-180, BYK-182, BYK-184, BYK-185, BYK-2050, BYK-2055, BYK-2015, BYK-9077 (manufactured by BYK Chemie), or the like.

An amount of the surfactant to be used is not particularly limited, and is, for example, 0.01 to 2% by mass, preferably 0.05 to 1% by mass with respect to the total of the components other than the solvent in the metal oxide dispersion, from the viewpoint of film-forming properties, coating properties, defoaming properties, and leveling properties of the metal oxide dispersion.

### [Other Ingredients]

Additives such as a dispersant, a thermal polymerization inhibitor, a defoaming agent, a silane coupling agent, a colorant (pigment or dye), a crosslinking agent, and an acid generating agent may be contained in the metal oxide dispersion according to the present invention, if necessary. As any of the additives, conventionally known additives can be used. Examples of the surfactant include anionic, cationic, and nonionic compounds, examples of the thermal polymerization inhibitor include hydroquinone and hydroquinone monoethyl ether, and examples of the defoaming agent include silicone compounds and fluorine compounds.

A method for producing the metal oxide dispersion according to the present invention is not particularly limited, and examples thereof include a method in which a carboxylic acid having 9 or more carbon atoms, metal oxide nanoparticles surface-treated with a capping agent, a solvent, optionally a surfactant, and optionally other components are uniformly mixed.

### <Method for Producing Metal Oxide Film>

A method for producing the metal oxide film according to the present invention includes a coating film forming step of forming a coating film including the metal oxide dispersion according to the present invention, and a heating step of heating the coating film at a temperature of 165° C. or higher.

The coating film can be formed, for example, by applying the metal oxide dispersion onto a substrate such as a semiconductor substrate. Examples of the coating method include a method using a contact transfer coating apparatus such as a roll coater, a reverse coater, a bar coater, etc. and a non-contact coating apparatus such as a spinner (rotary coating apparatus or spin coater), a dip coater, a spray coater, a slit coater, a curtain flow coater, etc. In addition, after adjusting the viscosity of the metal oxide dispersion to an appropriate range, the metal oxide dispersion may be applied by a printing method such as an inkjet method, a screen-printing method, etc. to form a coating film patterned into a desired shape.

The substrate preferably includes a metal film, a metal carbide film, a metal oxide film, a metal nitride film, or a metal oxynitride film. Examples of the metal constituting the substrate include silicon, titanium, tungsten, hafnium, zirconium, chromium, germanium, copper, aluminum, indium, gallium, arsenic, palladium, iron, tantalum, iridium, molybdenum, alloys thereof, etc. The substrate preferably includes silicon, germanium, and gallium. The surface of the substrate may have an uneven shape, and the uneven shape may be a patterned organic material.

Then, if necessary, volatile components such as a solvent are removed to dry the coating film. A drying method is not particularly limited, and examples thereof include a method of drying at a temperature of 80° C. or higher and 140° C. or lower, preferably 90° C. or higher and 130° C. or lower on a hot plate for a time period in a range of 60 seconds or more and 150 seconds or less. Before heating with a hot plate, vacuum drying may be performed at room temperature using a vacuum dryer (VCD).

After forming the coating film in this manner, the coating film is heated at a temperature of 165° C. or higher. The temperature during heating is not particularly limited, and is preferably 170° C. or higher, more preferably 175° C. or higher, and still more preferably 180° C. or higher. The upper limit may be appropriately set, and may be, for example, 600° C. or less, 550° C. or less, or 450° C. or less. Typically, the heating time is preferably 30 seconds or more and 150 seconds or less, and more preferably 60 seconds or more and 120 seconds or less. The heating step may be performed at a single heating temperature or may include multiple stages each having different heating temperatures.

Note that the thickness of the metal oxide film is not particularly limited as long as a desired effect is not impaired. The thickness of the metal oxide film is preferably 5 nm or more and 200 nm or less, more preferably 10 nm or more and 150 nm or less, still more preferably 20 nm or more and 100 nm or less. The thickness of the metal oxide film can be adjusted by adjusting the thickness of the coating film. For example, the film thickness of coating film can be adjusted by adjusting the solid content concentration and the viscosity.

The metal oxide film formed as described above is suitably used as, for example, a metal hard mask or a material for pattern inversion. Since the metal oxide film has excellent gap-fill characteristics, the metal oxide film can be easily formed as a planarization film on a substrate having a step, such as a substrate having a hole, a trench, or the like or a substrate provided with another member.

### EXAMPLES

Hereinafter, the present invention will be described in more detail with reference to Examples, but the present invention is not limited to these Examples.

### [Preparation of Metal Oxide Dispersions]

Undiluted dispersion solutions described below were prepared with reference to the description in paragraph of Japanese Unexamined Patent Application, Publication No. 2018-193481A.

### Preparation of Undiluted Dispersion Solution Z-1

According to the description in paragraph of Japanese Unexamined Patent Application, Publication No. 2018-193481A, a slurry of Zro2 obtained by cooling to room temperature was centrifuged to obtain a wet cake A. 2-acryloyloxyethyl phthalic acid in an amount of 0.25 times the weight of the wet cake A was added to the wet cake A and stirred. After reprecipitation, a wet cake B was obtained by centrifugation. The wet cake B was dried overnight under reduced pressure to obtain a powder. Propylene glycol monomethyl ether acetate (hereinafter, referred to as “PGMEA”) was added to the dry powder obtained so as to give a solid content concentration of 48% by mass, and the obtained mixture was redispersed and filtered to obtain an undiluted dispersion solution Z-1.

### Measurement of Size of Metal Oxide Nanoparticles Contained in Undiluted Dispersion Solution Z-1

Using the undiluted dispersion solution Z-1 as a sample, XRD measurement was performed using an X-ray diffractometer (SmartLab manufactured by Rigaku). The results obtained were analyzed by PDXL of the software attached, and the size (crystallite size) of the metal oxide nanoparticles was determined by a Halder-Wagner method and found to be 2.5 nm.

### Preparation of Carboxylic Acid Solutions

1.0 parts by mass of each of the carboxylic acids shown in Table 1 and 99 parts by mass of PGMEA were mixed to obtain carboxylic acid solutions A to F.

TABLE 1

| Carboxylic acid | Carboxylic acid | Solvent |
|---|---|---|
| solution | (1.0 part by mass) | (99 parts by mass) |
| A | Lauric acid | PGMEA |
| B | Stearic acid | PGMEA |
| C | n-octyl succinic acid | PGMEA |
| D | 4-(hexyloxy)benzoic acid | PGMEA |
| E | 12-hydroxystearic acid | PGMEA |
| F | 2-ethylhexanoic acid | PGMEA |

### Preparation of Surfactant Solution

0.6 parts by mass of a commercially available surfactant (BYK-164 (trade name) manufactured by BYK Chemie) and 99.4 parts by mass of PGMEA were mixed to obtain a surfactant solution.

### Preparation of Metal Oxide Dispersions

At a ratio (unit:part(s) by mass) shown in Table 2, each of the carboxylic acid solutions and the surfactant solution were added to the undiluted dispersion solution Z-1, and then solvent PGMEA was added, and the obtained mixture was stirred and filtered through a membrane filter having a diameter of 0.2 μm to obtain a metal oxide dispersion.

### [Ratio of Inorganic Component Mass]

In Table 2, “Ratio of inorganic component mass” indicates a ratio of the inorganic component mass to a total of the inorganic component mass and an organic component mass, with respect to the solid content of the metal oxide dispersion. Specifically, a ratio (mass %) of a mass of the inorganic component in the dispersion Z-1 to a total of a mass of the carboxylic acid in the carboxylic acid solution, a mass of solid content of the dispersion Z-1, and a mass of the surfactant in the surfactant solution was calculated.

### [Ratio of Free Carboxylic Acid]

In Table 2, “Ratio of free carboxylic acid” indicates a ratio of a carboxylic acid present in the metal oxide dispersion independently from the metal oxide nanoparticles, relative to the mass of the solid content of the metal oxide dispersion. Specifically, a ratio (mass %) of a mass of the carboxylic acid in the carboxylic acid solution to a total of a mass of the carboxylic acid in the carboxylic acid solution, a mass of solid content of the dispersion Z-1, and a mass of the surfactant in the surfactant solution was calculated.

### [Evaluation of Dispersibility]

A metal oxide dispersion immediately after the preparation was used as a sample. The sample left to stand for 24 hours was visually observed, and the dispersibility was evaluated according to the following criteria. The results are shown in Table 2.
+ (good): the metal oxide nanoparticles were dispersed in the sample after standing.
− (poor): the metal oxide nanoparticles had settled in the sample after standing.

### [Preparation of Metal Oxide Film]

The metal oxide dispersion was added dropwise onto a 6-inch silicon wafer having a trench with a width of 50 nm and a depth of 50 nm, and spin coating was performed at 750 rpm for 30 seconds. Thereafter, pre-baking was performed at 100° C. for 120 seconds using a hot plate, and post-baking was performed at the following firing temperatures for 90 seconds to obtain a metal oxide film having a film thickness of about 30 nm. Note that the thickness of the metal oxide film was measured by observing a cross section of the metal oxide film with an SEM. Firing temperature: 150° C., 180° C., and 200° C.

### [Evaluation of Dry State]

The metal oxide film was visually observed, and the dry state of the metal oxide film was evaluated according to the following criteria. The results are shown in Table 2.
+ (good): the solvent was completely volatilized from the metal oxide film.
− (poor): the solvent remained in the metal oxide film.

### [Evaluation of Gap-fill Characteristics]

The cross section of the metal oxide film at a portion where the trench was formed was observed by SEM, and the gap-fill characteristics were evaluated according to the following criteria. The results are shown in Table 2.
+ (good): the metal oxide film was uniformly embedded in the trench.
− (slightly poor): relatively fewer voids were observed in the trench.
−− (poor): a relatively large number of voids were observed in the trench.

TABLE 2

| | | | | | | | Comparative | |
|---|---|---|---|---|---|---|---|---|
| | | Example | | | | | Example | |
| | | 1 | 2 | 3 | 4 | 5 | 1 | 2 |
| Ratio of inorganic | | 71.58 | 71.58 | 71.58 | 71.58 | 71.58 | 71.58 | 79.50 |
| component mass | | | | | | | | |
| Ratio of free | | 9.96 | 9.96 | 9.96 | 9.96 | 9.96 | 9.96 | 0.00 |
| carboxylic acid | | | | | | | | |
| Carboxylic | A | 4.97 | | | | | | |
| acid solution | B | | 4.97 | | | | | |
| | C | | | 4.97 | | | | |
| | D | | | | 4.97 | | | |
| | E | | | | | 4.97 | | |
| | F | | | | | | 4.97 | |
| Undiluted | Z-1 | 0.93 | 0.93 | 0.93 | 0.93 | 0.93 | 0.93 | 1.04 |
| dispersion | | | | | | | | |
| Surfactant solution | | 0.47 | 0.47 | 0.47 | 0.47 | 0.47 | 0.47 | 0.52 |
| Solvent | PGMEA | 3.63 | 3.63 | 3.63 | 3.63 | 3.63 | 3.63 | 8.44 |
| Total | | 10 | 10 | 10 | 10 | 10 | 10 | 10 |
| Dispersibility | | + | + | + | + | + | + | + |
| Gap-fill | After | + | + | + | + | + | + | − |
| characteristic | pre-baking | | | | | | | |
| Dry state | After | + | + | + | + | + | + | + |
| | post-baking | | | | | | | |
| | at 200° C. | | | | | | | |
| Gap-fill | After | + | + | + | + | + | −− | −− |
| characteristic | post-baking | | | | | | | |
| | at 200° C. | | | | | | | |
| Dry state | After | + | + | + | + | + | + | + |
| | post-baking | | | | | | | |
| | at 180° C. | | | | | | | |
| Gap-fill | After | + | + | + | + | + | − | − |
| characteristic | post-baking | | | | | | | |
| | at 180° C. | | | | | | | |
| Dry state | After | − | − | − | − | − | − | − |
| | post-baking | | | | | | | |
| | at 150° C. | | | | | | | |
| Gap-fill | After | + | + | + | + | + | + | + |
| characteristic | post-baking | | | | | | | |
| | at 150° C. | | | | | | | |

As can be seen from Table 2, it was confirmed that the metal oxide dispersions of the Examples were excellent in the gap-fill characteristics, whereas the metal oxide dispersions of the Comparative Examples were inferior in the gap-fill characteristics.

1. A metal oxide dispersion comprising:
a carboxylic acid having 9 or more carbon atoms;
metal oxide nanoparticles surface-treated with a capping agent, and
a solvent.

2. A metal oxide dispersion comprising:
a carboxylic acid having a boiling point of 250° C. or higher at atmospheric pressure;
metal oxide nanoparticles surface-treated with a capping agent; and
a solvent.

3. The metal oxide dispersion according to claim 1, further comprising a surfactant.

4. The metal oxide dispersion according to claim 1,
wherein, in a solid content of the metal oxide dispersion, a ratio of an inorganic component mass to a total of the inorganic component mass and an organic component mass is 25 mass % or more.

5. The metal oxide dispersion according to claim 1, wherein the metal contained in the metal oxide nanoparticles is at least one type selected from the group consisting of zinc, yttrium, hafnium, zirconium, lanthanum, cerium, neodymium, gadolinium, holmium, lutetium, tantalum, titanium, silicon, aluminum, antimony, tin, indium, tungsten, copper, vanadium, chromium, niobium, molybdenum, ruthenium, rhodium, rhenium, iridium, germanium, gallium, thallium, and magnesium.

6. A method for manufacturing a metal oxide film comprising:
forming a coating film comprising the metal oxide dispersion according to claim 1; and
heating the coating film at a temperature of 165° C. or higher.

7. The method for manufacturing a metal oxide film according to claim 6, wherein the metal oxide film is formed as a planarization film.

8. The metal oxide dispersion according to claim 2, further comprising a surfactant.

9. The metal oxide dispersion according to claim 2,
wherein, in a solid content of the metal oxide dispersion, a ratio of an inorganic component mass to a total of the inorganic component mass and an organic component mass is 25 mass % or more.

10. The metal oxide dispersion according to claim 2, wherein the metal contained in the metal oxide nanoparticles is at least one type selected from the group consisting of zinc, yttrium, hafnium, zirconium, lanthanum, cerium, neodymium, gadolinium, holmium, lutetium, tantalum, titanium, silicon, aluminum, antimony, tin, indium, tungsten, copper, vanadium, chromium, niobium, molybdenum, ruthenium, rhodium, rhenium, iridium, germanium, gallium, thallium, and magnesium.

11. A method for manufacturing a metal oxide film comprising:
forming a coating film comprising the metal oxide dispersion according to claim 2; and
heating the coating film at a temperature of 165° C. or higher.

12. The method for manufacturing a metal oxide film according to claim 11, wherein the metal oxide film is formed as a planarization film.
